(19) United States
(12) Patent Application Publication
(10) Pub. No.: US 2022/0068447 A1
(43) Pub. Date: Mar. 3, 2022

(54) Personal care management system and method

(71) Applicant: Boyd Soussana, Saint-Lazare (CA)

(72) Inventors: Boyd Soussana, Saint-Lazare (CA); David Turner, St.Catharines (CA)

(21) Appl. No.: 17/008,250

(22) Filed: Aug. 31, 2020

(51) Int. Cl. G16H 10/65; G16H 40/67; G16H 40/40; G16H 20/10; G16H 80/00

(52) U.S. Cl. CPC G16H 10/65; G16H 40/67; G16H 80/00; G16H 20/10; G16H 40/40

(57) ABSTRACT

A system and method are provided to coordinate the delivery of the different services provided by healthcare professionals and personal care workers, or personal support workers (PSWs). The invention provides a fast and easy means of documenting, in real-time, patient information that is uploaded and immediately accessible remotely to other health professionals involved in the patient care. It is the first electronic health record to integrate a quick response (QR) code tailored for in-home care. The systems/methods incorporate patients, medical care providers and PSWs. The systems and methods include: 1) a patient portal, which allows patients to interact with the system; 2) a provider portal which serves as a medium in which providers and PSWs interact with the system; and 3) an administration portal.

### CROSS-REFERENCE TO RELATED APPLICATIONS

The present application claims the benefits of priority of U.S. Provisional Patent Application No. 62/894,408, entitled “PERSONAL CARE MANAGEMENT SYSTEM AND METHOD”, and filed at the United States Patent and Trademark Office on Aug. 30, 2019, the content of which is incorporated herein by reference.

### FIELD OF THE INVENTION

The invention relates generally to medical information storage and retrieval systems and, more particularly, to techniques for conveying medical and visit information when a personal support worker visits an in-home patient using quick response codes.

### BACKGROUND OF THE INVENTION

Home care is becoming more and more a key part of the health care systems throughout the world as governments try to deal with the rapidly increasing age of the population and the associated rising costs of providing health care services in hospitals and other health care facilities. The management and sharing of health information is critically important in home care due to the involvement of multiple health professionals including physicians, nurses, physiotherapists, and other home care personnel (Koch et al., 2004). This has resulted in the development of multiple strategies to gather and manage information in this setting. Electronic health information systems (EHIS) are integrated, computer-assisted systems that are able to collect, store, and organize comprehensive patient information (World Health Organization, 2005; Stolee et al., 2010). In addition, governments and insurers around the world are now grappling with how to track and verify, in real time, the budgets allocated for in-home patients care and whether they are actually going for the services received by the in-home care patient. The patient-centered medical home has been promoted as a model of improved care that addresses many of the failures and delivery gaps within the current primary care system (Bates and Bitton, 2010). Unfortunately, there continue to be limitations in the way home care organizations manage and use information, which lead to inadequate provision of services. Better use and management of information will lead to major benefits for the health, quality of life, and independence of older persons receiving home care, while also providing important system benefits through decreased costs, more efficient use of resources and avoiding unnecessary and expensive institutional placements.

One major challenge is how to coordinate the delivery of the different services provided by healthcare professionals and personal care workers, or personal support workers (PSWs). Adoption of electronic health records (EHR), or electronic medical records (EMD) have the potential to significantly improve the overall quality of healthcare delivered to in-home patients by addressing some of these failures. One major pitfall of current technologies is the lack of patient involvement in the process. Opportunities to obtain critical information directly from the patient, in real-time, about key parameters such as weight, blood pressure and/or blood sugars using these technologies are largely missed. Also, an estimated 12 percent of patients suffer from an adverse drug event after being discharged from the hospital (Forster et al., 2003). Communication technologies targeting in-home patients can definitely mitigate this risk by integrating prescription regimens and coordinating an in-home visit by a health professional such as a nurse. Among the other pitfalls of existing technologies are their complexity (not user-friendly), their incompatibly with existing systems, their lack of adaptability for changing regulations in the healthcare system and, most importantly, patient-confidentiality.

The present invention seeks to solve these issues, among others, while providing comfort and peace of mind to the loved ones of an in-home patient that they are receiving the services the providers have said they would receive. The present invention provides a fast and easy means of documenting, in real-time, patient information that is uploaded and immediately accessible remotely to other health professionals involved in the patient care. The present invention comprises a novel EHR which integrates a quick response (QR) code tailored for in-home care as well as a novel medical record system using a quick response code's functionality targeted to in-home patients and home-care providers. Finally, the present invention seeks to ensure patient-confidentiality by conforming to the fair information practices (FIPs) principles.⁴

### SUMMARY OF THE INVENTION

Various embodiments illustrated herein relate to a system for storing patients' medical records electronically and provide a computerized platform, including user-friendly software applications for personal computers and mobile devices. The user-friendly software application is compatible with most existing systems. The interoperability of the present invention with existing systems seeks to provide a smooth and progressive adoption of the invention with minimal risks associated with high transition costs and the general reluctance of health professionals to implement new communication technologies.

In one embodiment, the system and method include a patient interface, referred to herein as the patient portal.

In another embodiment, the system and method include a health professional interface, referred to herein as the provider portal; the health professional including, but not limited to, primary physicians, specialized physicians, mental health providers (ex: psychiatrists, psychologists and social workers), pharmacists, nurses, physiotherapists, chiropractors and researchers.

In another embodiment, the system and method include an administrative personnel interface, referred to herein as the administration portal; the administrative personnel including, but not limited to, administrative personnel, government agencies, personal support workers, health insurers, family and friends.

In another embodiment, each health professional will have access to specific information in accordance with fair information practices and the patient's approval of information sharing between professionals.

In another embodiment, the medical information electronically stored include, but is not limited to, the patient's contact information and emergency contact information, photograph, age, language(s), blood type, allergies, immunization record, medical devices used, past illnesses and surgeries, chronic conditions, current medical condition, past and current medications (prescription and over-the-counter), test results, mental health information and genetic information.

In yet another embodiment, the systems are further enhanced by the use of a computer system for generating quick response (QR) codes for each patient.

In another embodiment, a database is connected to a system server that is connected to a publicly accessible network such as the Internet.

In yet another embodiment of the current invention, the user-friendly software is a mobile application for scanning the QR code located on the patient's EMS 24/7 Medical ID Card at the beginning and at the end of a patient visit.

In another embodiment, a speech-to-text functionality is integrated in the software for the care provider to easily document details of an in-home visit hands-free.

In another embodiment, an integrated translator function can facilitate communication when a language barrier is an issue.

In another embodiment, a telehealth technology allows the in-home patient to be monitored at distance by the healthcare professional.

In one embodiment, coordination between the patient, the healthcare provider and the health insurer will be facilitated with claim submissions made via the portal.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1. discloses an overview of the elements involved with the present invention.

FIG. 2. discloses an example of a display screen for patients to access their medical information in accordance with the present invention.

FIG. 3. discloses a second example of a display screen for patients to update their medical information that will be available upon scanning of the QR code on the EMS 24/7 Global ID card in accordance with the present invention.

FIG. 4. discloses a medical ID card in accordance with the present invention.

FIG. 5. discloses an example of a display screen allowing the patient to modify the sharing preferences with respect to their medical information accordance with the present invention.

FIG. 6. discloses an example of a display screen allowing the patient to select the medical information they wish to share with a chosen contact in accordance with the present invention.

FIG. 7. discloses an example of a display screen for the healthcare professional to view their upcoming appointments in accordance with the present invention.

FIG. 8. discloses an example of a display screen for administrative staff or government agency that tracks the date, time and location of visits by the personal support worker, in accordance with the present invention.

FIG. 9. Discloses an example of a display screen for patients comprising a calendar to view their upcoming or past visits, in accordance with the present invention.

FIG. 10. discloses an example of a display screen for the patient to view the details of their assigned personal support worker in accordance with the present invention.

FIG. 11. discloses an example of a display screen once the QR code is scanned by a QR coder reader. The barcode reader can be an app on a mobile device such as an iPhone or any Android-operated mobile device. By scanning the code using the app, the mobile device only displays the critical medical data the patient has chosen, as depicted in FIG. 6. and in accordance with the present invention.

FIG. 12. discloses the Prior Art in accordance with the present invention

### DETAILED DESCRIPTION OF THE PREFERRED EMBODIMENT

The novel personal care management system and method (PCMS), also referred to as the Personal Care Tracker™, will be described hereinafter. Although the invention is described in terms of specific illustrative embodiments, it is to be understood that the embodiments described herein are by way of example only and that the scope of the invention is not intended to be limited thereby.

The systems and methods described herein incorporate patients, medical care providers and personal support workers (PSWs), all of which contribute to a patient visit, personal care record generation and revision, and a patient's personal feelings about the visit. The PCMS 100 includes three aspects, which are referred to herein as portals: 1) a patient portal 200, which allows patients to interact with the system; 2) a provider portal 300 which serves as a medium in which providers and PSWs interact with the system; and 3) an administration portal 400. The portals may be accessed by means of a website, a mobile application, a desktop application or any other application software suitable for interfacing with a computer program.

In certain embodiments, the provider portal 300 may be accessible by PSWs or healthcare professionals including, but not limited to, primary physicians, specialized physicians, mental health providers (ex: psychiatrists, psychologists and social workers), pharmacists, nurses, physiotherapists, chiropractors and researchers. Similarly, the administration portal 400 may be made available to the administrative personnel including, but not limited to, administrative personnel, government agencies, personal support workers, health insurers, family and friends. In a preferred embodiment, access to the provider portal 300 and administration portal 400 will be granted in accordance with fair information practices and the patient's approval of information sharing between professionals.

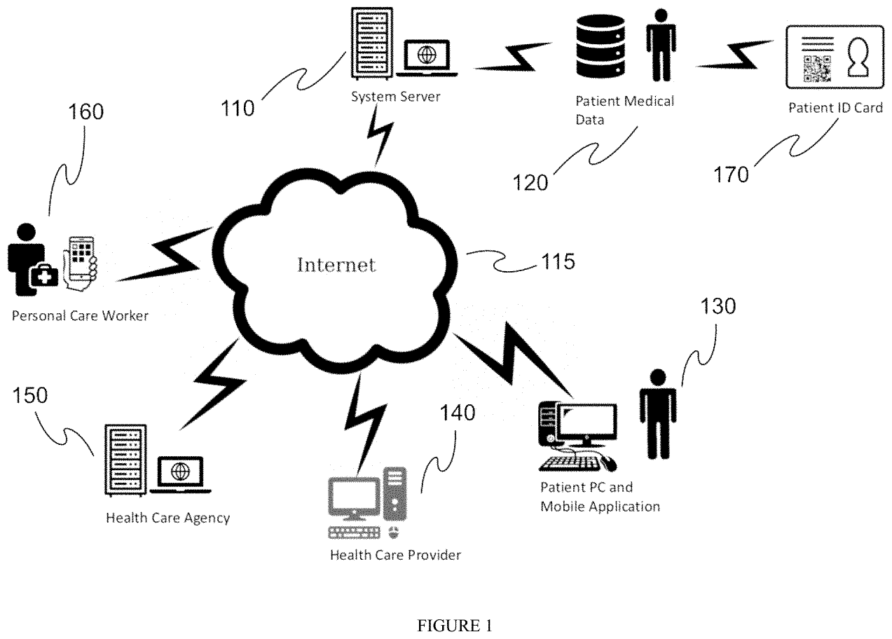

In certain embodiments, the PCMS 100 includes a plurality of personal computer systems and mobile applications for a plurality of healthcare providers, administrative staff and the patient. The computer systems may comprise computers (PCs), handheld devices/personal digital assistants (PDAS), or other browser-enabled appliances or devices. In the example embodiment depicted in FIG. 1, the PCMS 100 includes a remote server computer system 110 linked by the internet 115 or other communication channels to the other computer systems. In other embodiments, the PCMS 100 may comprise multiple remote servers 110. The PCMS 100 may further allow connections between one or more computer systems consisting of any combination of one or more patient applications 130, health care provider system 140, health care agency system 150 and/or personal care worker system 160.

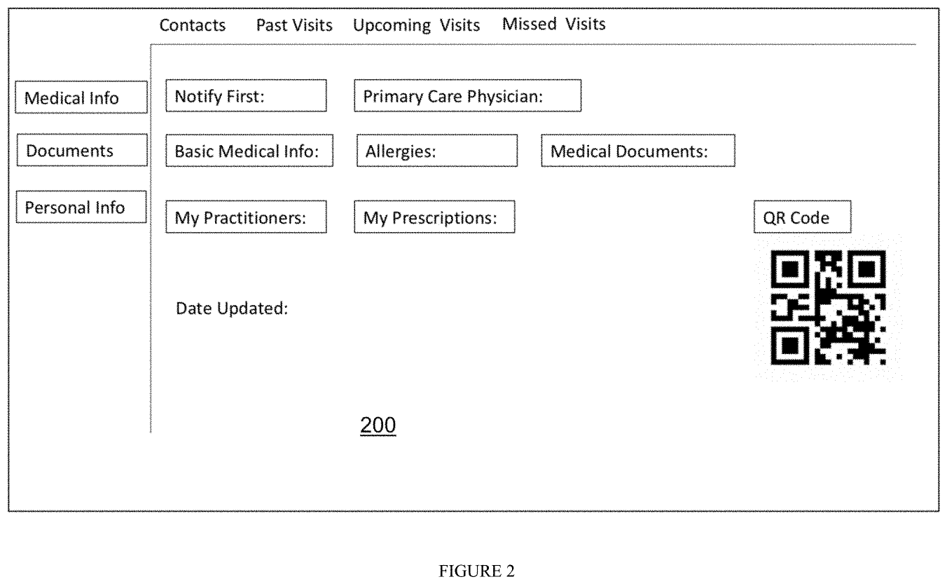

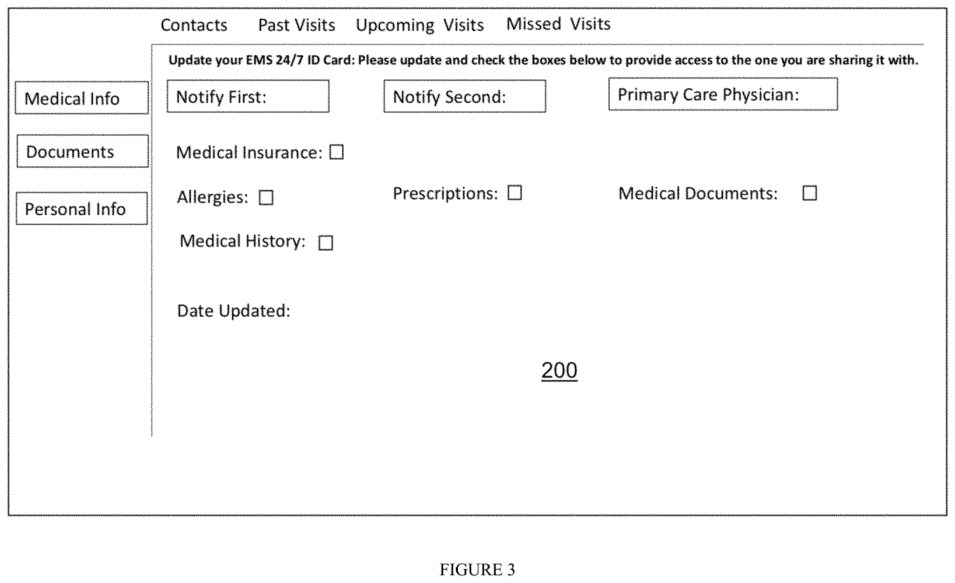

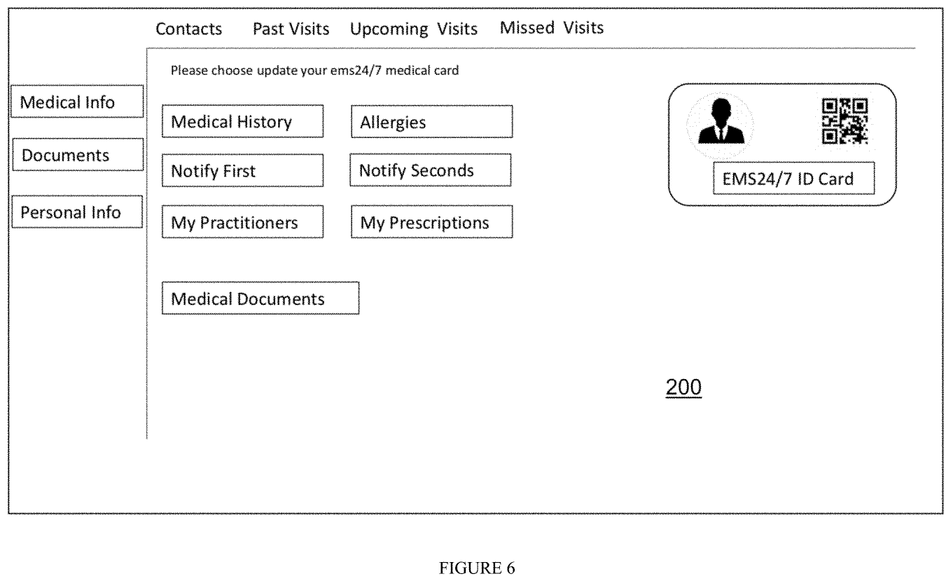

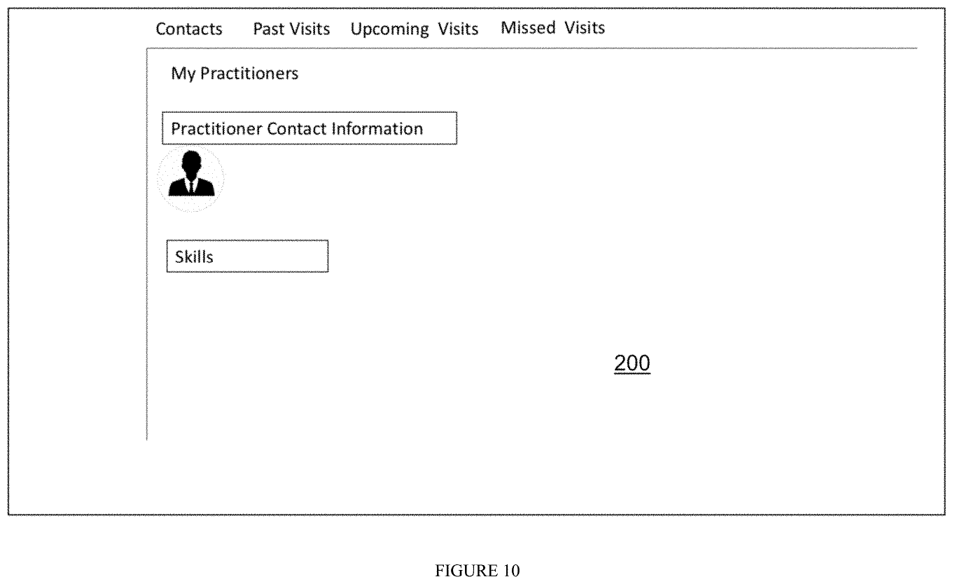

The computer systems are known to include processors running operating systems, storage mediums, input devices and an outputs, the interfaces of which are illustrated in FIGS. 2-3, 5-11 as the user interfaces, or portals. The PCMS 100, as depicted in FIG. 1, allows medical information to be integrated from all computer systems. The computer systems may further communicate with the remote server 110 via the world wide web using the Hypertext Transfer Protocol (HTTP) whereas the remote server 110 gives users access to encrypted and password/biometric-protected files. Files can be in different text, graphic, audio and video format using the well-known page description language Hypertext Markup Language (HTML).

In certain embodiments, the files uploaded and/or stored on the remote server 110 consist of patient medical data 120 thereby allowing access to the patient medical data 120 by means of any of the aforementioned portals. The remote server 110 may therefore allow synchronization between computer systems and automatic software updates, in the background, without any required actions by the user. The patient medical data 120 may be read-only to protect both the patient and the healthcare professional from alterations in order to prevent unauthorized modifications. In addition, the PCMS 100 may be configured to record modifications, whether authorized or unauthorized, with a log of the time and source of the modification.

Moreover, the patient medical data 120 consisting of paper files which are uploaded may be converted into digital form to make them content therein searchable by a user and documents may be sorted and indexed appropriately for easy retrieval. The classification fields may include, but are not limited to, date, healthcare provider, location, reason of visit, CPT (billing code), type of record, type of test, current and past prescriptions. Moreover, all patient medical data 120 may be encrypted using an encryption algorithm. In other embodiments however, patient medical data 120 may be selectively exempt from encryption. Such patient medical data 120 may include contact information and emergency contact information, photograph, age, language(s), blood type, allergies, immunization record and medical devices used.

It may be appreciated that the present invention may provide patients an ease with consulting a secondary physician and obtaining a second opinion related to their diagnostic and treatment strategy without the burden of having to ask their primary care provider to transfer the patient medical data 120 to the second physician.

The PCMS 100 may additionally be configured to selectively limit access to the personal medical data 120 of a patient. Thus, a PSW will not have access to the patient medical data 120 as the primary physician of the patient who will likely have access to all, or almost all, of the patient medical data 120. Similarly, a pharmacist will have access to certain patient medical data 120 relevant to his practice such as information on the disease state and medication history. Among the information uploaded to a central database, or remote server 110, and filtered onto the patient portal 200 is the patient's current medical condition, past and current medications (prescription and over-the-counter), test results, mental health information and genetic information. The patient medical data 120 being less sensitive such as the patient's contact information and emergency contact information, photograph, age, language(s), blood type, allergies, immunization record and medical devices used will likely be available to all healthcare care providers and administrative staff.

In a preferred embodiment, the user/patient and the PSW are registered on the PCMS 100. To that end, the user/patient is issued a username and temporary password and logs into the PCMS 100. The user/patient accesses the patient portal 200 and can populate their own information within the PCMS 100.

Referring now FIGS. 2-3 and 5-11, example embodiments of software applications, or portals, of the PCMS 100 are now shown. The portals seek to integrate easy to use and intuitive functionalities allowing for minimal training on the part of health professionals while encouraging consistent use by patients.

More specifically and with reference to FIGS. 2, 3, 5 and 6, an example embodiment of the patient portal 200 operating as part of the patient application 130 is shown. In this example embodiment, the portal 200 offers the patient a platform to access their medical information, their prescriptions, upcoming appointments and any other relevant data. The patient portal 200 may comprise an enhanced interface tailored to the patient's needs. For example, the patient portal 200 may include a voice recognition function to allow a patient to document their current symptoms, in real-time, hands-free. In other embodiments, the patient portal 200 may be prompt the user to input information regarding their status or condition. These prompts may additionally be personalized based on the patient's medical history such that only relevant questions related to their disease state are presented. For example, if a patient is diabetic, the patient portal 200 will ask to log their weight, activity levels, frequency of urination, liquid and food intake as well as overall feelings of fatigue, thirst and hunger. Using input data and regularly updated medical information, the patient portal 200 may be able to predict, based on the patient's history and real-time symptoms, if a change in treatment strategy or an emergency visit from a nurse is necessary or, as a last resort, if hospitalization would be the better option.

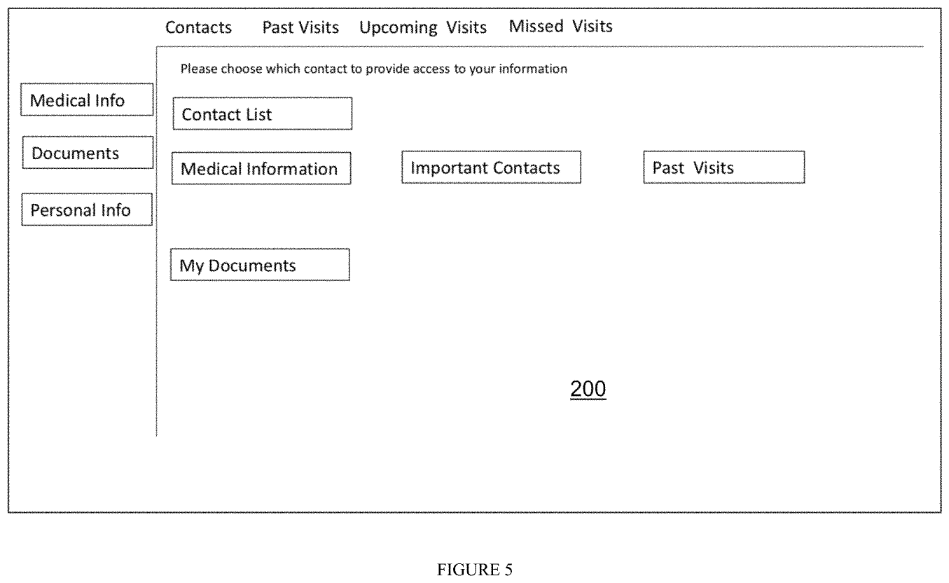

In certain embodiments and as shown in FIG. 5, the patient portal 200 may be configured to allow a patient to also decide what patient medical data 120 is made available to each healthcare provider. The patient can choose to share additional data from the patient medical data 120 with the professional if they choose to. It may be appreciated that this personal engagement may provide the patient with a sense of empowerment and control over the care they receive.

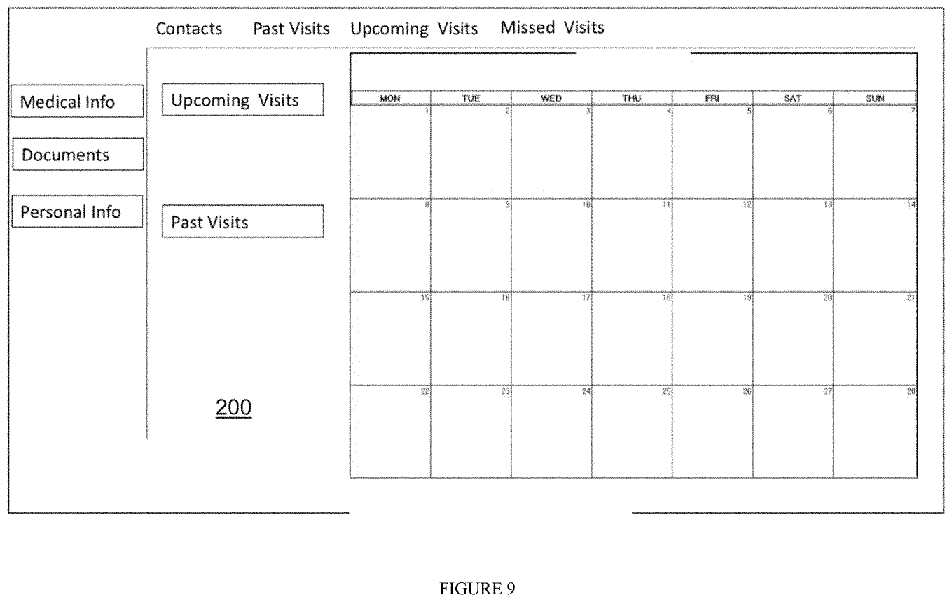

Referring now to FIG. 9, the patient portal 200 may additionally comprise a calendar configured to allow patients to setup or view appointments (past or upcoming). In certain embodiments, the patient application 130 may comprise notifications for upcoming appointments, reminders to take medications and a checkbox to keep track of medications taken. Other notifications will take the age of the patient and the last screening exam and notify the patient and health professional when another exam is due for scheduling an appointment.

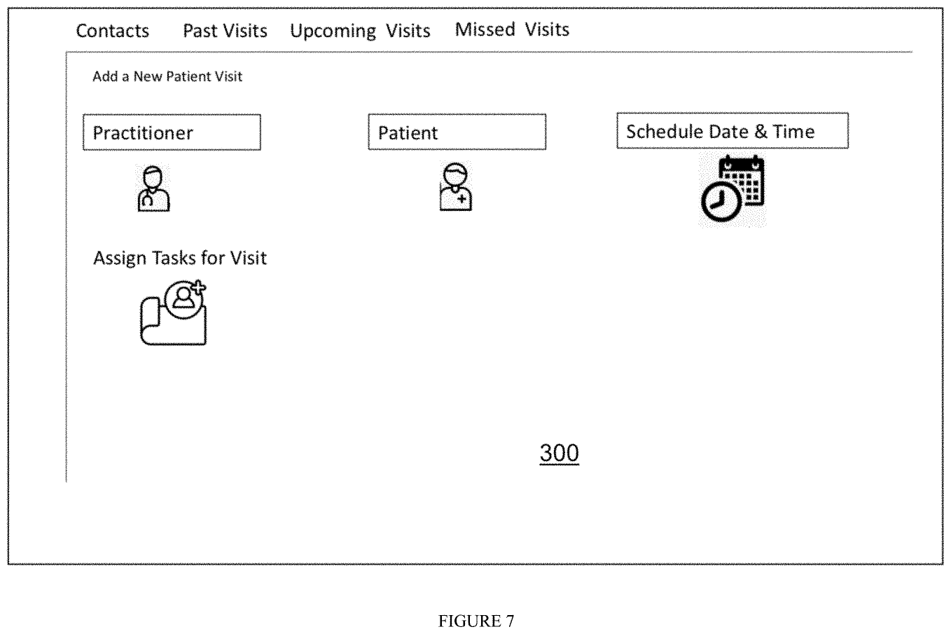

The computer systems consisting of health care provider systems 140 may have access to the provider portal 300 tailored to them so as to make the relevant information easily accessible and minimize the burden of sorting through irrelevant details, in addition to protecting patient confidentiality as per the FIPs. As such and as shown in FIG. 7, the provider portal 300 may offer health care provider systems 140 the ability to uploading and downloading/viewing patient medical data 120.

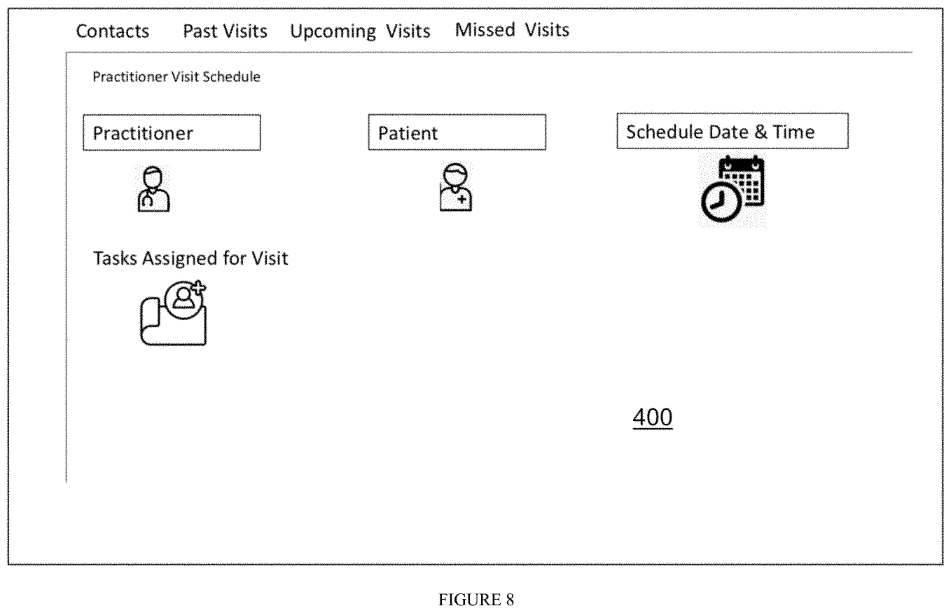

In certain embodiments, the administrative portal 400 may serve as a medium in which the health care agency systems 150 and the personal care worker systems 160 interact with each other and the patient. FIG. 8 illustrates an embodiment of an administrative portal 400 allowing for the scheduling of appointments, practitioners, tasks or access any other required data. The administrative portal 400 may additionally provide users statistical data regarding individual patients or global trends thereby allowing government agencies or health organizations to derive in depth healthcare information.

Although not described in detail herein, the PCMS 100 may comprise additional portals allowing access to other forms of administrative support staff, personal support workers, family, friends and health insurers. Since research shows a reluctance on the part of patients to share sensitive information with their health insurers, the patient medical data 120 included in these portals may be limited by default (Caine & Hanania, 2013).

The PCMS 100 may be further enhanced by the use of an automatic identification tag to allow PSW, healthcare professionals or any other users to easily and quickly identify and authenticate a patient.

In certain embodiments, the automatic identification tag may comprise a quick response (QR) code, an NFC tag, a keycard or any other suitable means of automatic identification. In other embodiments, the automatic identification tag may comprise a biometric measurement of the patient. For example, the biometric measurement may comprise a fingerprint reading, a retinal scan, a facial scan, a DNA scan or any other suitable biometric measurement.

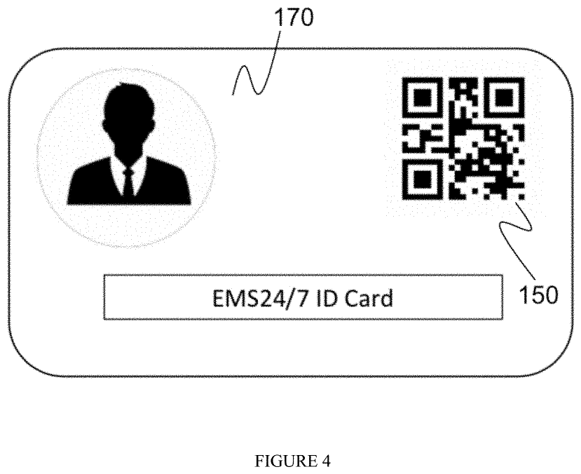

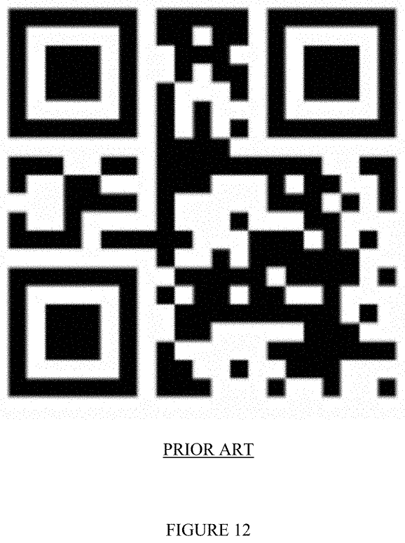

In the present embodiment, the automatic identification tag comprises a QR code 500. A prior art example of a QR code 500 is shown in FIG. 12. To that end, the PCMS 100 may comprise a computer system for generating quick response (QR) codes 500 for a patient. The scanning of the QR code 500 by a PSW or healthcare professional using an application of the PCMS 100 allows for tracking of the date, time of arrival, time of departure and location of the visit with the patient. Referring now to FIG. 4, a patient's medical ID card 170 is shown. In this example, the medical ID card 170 comprises the QR code 500 which serves as the automatic identification tag.

In practice, an in-home visit of the patient may be initiated by a health care provider system 140 or a health care agency system 150. The patient application 130 and personal care worker system 160 may subsequently receive an appointment confirmation of the in-home visit. The notification may indicate the date, time and location of the appointment. The appointment confirmation may comprise an email, a notification or any other suitable confirmation.

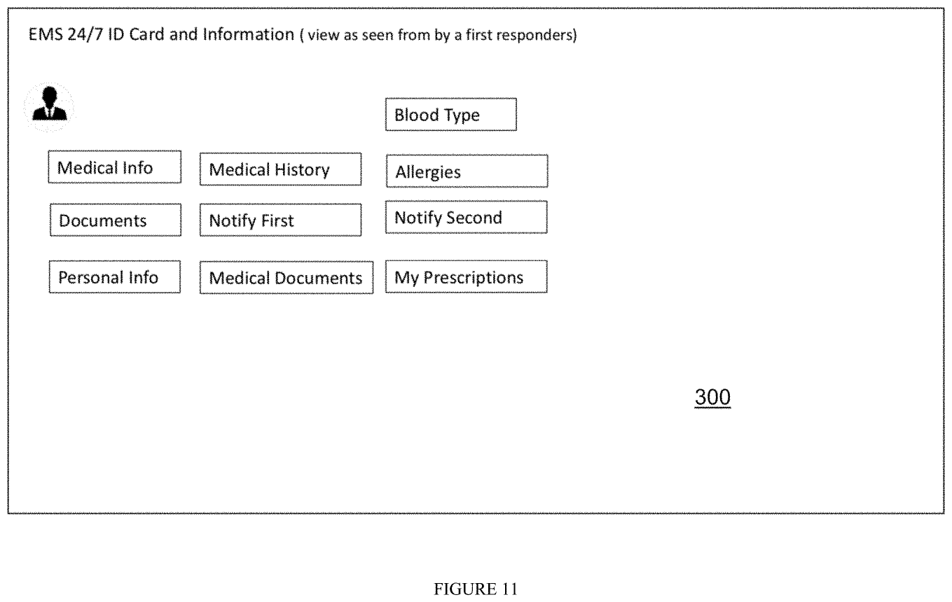

In certain embodiments, a PSW or healthcare professional may log into a provider portal 300 on the personal care worker system 160 and view all assigned patients and the corresponding visit calendar that is initiated by the health care provider system 140. The PSW may use the application of the PCMS 100 to scan the QR code 500 located on the patient's EMS 24/7 medical ID card 170. The QR code 500 may be scanned once the PSW or healthcare professional arrives at an appointment, at the the end of a visit of a patient visit or at any other suitable time. Referring now to FIG. 11, the provider portal 300 may illustrate the relevant medical data 120 when the QR code 500 is scanned.

In certain embodiments, the personal care worker system 160 comprises integrated support tools. These integrated support tools may be accessible on a mobile device to assist the health care provider in making decisions regarding treatment strategies. Moreover, the support tools may integrate all the patient medical history and improve over time as the patient enters more data such as objective physiological input, for example blood pressure, heart rate, weight as well as their biochemistry results following blood tests. In addition, patient medical data 120 consisting of subjective input data such as feelings of overall well-being and pain assessment will be recorded over time and will be able to propose causes and solutions to deteriorating conditions. This may help the health care provider to consider all relevant variables and ensure that none are forgotten.

In certain embodiments, the PCMS 100 mobile application will issue to the personal care worker system 160 timed messages to verify that the PSW is still with the patient and may automatically close or end the visit should the PSW move once predetermined conditions are met. For example, the visit may be closed or ended once the PSW is geolocated outside a predetermined area away from the initial scan (i.e. 100 m²) or if the personal care worker system 160 detects a traveling speed being above a predetermined threshold (i.e. above 3 km/h).

In yet another embodiment, the patient medical data 120 may be made available, anonymously, to researchers. This may be especially relevant for pharmaceutical companies who can monitor, using these large datasets, the safety and efficacy as well as any adverse events related to a medication dosage regimen together with the patient's personal information, putting safeguards to prevent full disclosure of the patient's identity.

It may be appreciated that the disclosed embodiments provide multiple benefits, including a fully accountable centralized, real-time electronic record of the patient visit to which a patient, a patient's caregivers, the health care provider and the Government health agencies can gain access from any web-enabled computer.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

### REFERENCES

Bates, D. W. and Bitton, A. (2010) The future of health information technology in the patient-centered medical home. Health Affairs, 29(4) 614-621.
Caine, K. & Hanania, R. (2013) Patients want granular privacy control over health information in electronic medical records. J Am Med Inform Assoc, 20, 7-15.
Forster A J, Murff H J, Peterson J F, Gandhi T K, Bates D W. The incidence and severity of adverse events affecting patients after discharge from the hospital. Ann Intern Med. 2003; 138(3):161-7.
Koch, S., Hagglund, M., Scandurra, I., & Mostrom, D. (2004). Towards a virtual health record for mobile home care of elderly citizens. Studies in Health Technology and Informatics, 107(Pt. 2), 960-963.
Office of the National Coordinator for Health Information Technology, & U.S. Department of Health and Human Services. Nationwide Privacy and Security Framework For Electronic Exchange of Individually Identifiable Health Information. 2008. Retrieved from http://healthit.hhs.gov/portal/server.pt/community/healthit_hhs_gov_privacy_security_framework/1173
Stolee, P., Steeves, B., Glenny, C. & Filsinger, S. (2010) The use of electronic health systems in home care. Home Healthcare Nurse, 28(3), 167-181.
World Health Organization. (2005). Bulletin of the World Health Organization (BLT), 83(8), 561-640.

1) A system for managing the personal care of a patient comprising:
at least one server having a memory in which patient medical data associated to the patient is stored;
a patient computer system in communication with the server operable to communicate and receive health care information with the patient;
one or more healthcare professional systems in communication with the server operable to view patient medical data and to provide health care information;
wherein the healthcare professional systems identify a patient and access the patient medical data by means of an automatic identification tag assigned to the patient.

2) The system of claim 1, wherein the server is configured to synchronize information between the patient computer system and the one or more healthcare professional systems.

3) The system of claim 1, wherein a portion of the patient medical data cannot be electronically modified.

4) The system of claim 1, wherein the patient medical data is configured to be shared with another healthcare professional system to obtain a second opinion.

5) The system of claim 1, wherein the patient computer system comprises an enhanced interface tailored to a patient's needs.

6) The system of claim 5, wherein the enhanced interface comprises a voice recognition function.

7) The system of claim 1, wherein one or more of the healthcare professional systems comprises a provider portal configured to allow access to a first set of the patient medical data.

8) The system of claim 7, wherein one or more of the healthcare professional systems comprises an administrative portal configured to allow access to a second set of the patient medical data

9) The system of claim 1, wherein the automatic identification tag is located on a medical ID card.

10) The system of claim 1 further comprising an appointment scheduling system.

11) The system of claim 10, wherein the scheduling system is configured to send a notification to the patient computer system and the one or more healthcare professional systems.

12) A method of managing the personal care of a patient comprising:
a. identifying a patient using an automatic identification tag;
b. remotely storing patient medical data associated to the identified patient;
c. remotely providing one or more healthcare professionals access to the patient medical data;
d. remotely providing the patient access to the patient medical data;
e. remotely providing the patient access to healthcare information generated by the healthcare professionals.

13) The method of claim 12 further comprising prompting the patient to input data based on their current symptoms in response to predetermined questions.

14) The method of claim 13 further comprising personalizing the questions based on the patient's medical history.

15) The method of claim 12 further comprising scheduling an appointment between the patient and the one or more healthcare professionals.

16) The method of claim 15, wherein the one or more healthcare professionals scan the automatic identification tag associated to the patient to confirm their arrival at the appointment.

17) The method of claim 16 further comprising automatically detecting when the one or more healthcare professionals have left the appointment.

18) The method of claim 17, wherein the departure of the one or more healthcare professionals is detected based on their geolocation.

19) The method of claim 17, wherein the departure of the one or more healthcare professionals is detected based on their traveling speed.

20) (canceled)

21) The method of claim 12 further comprising reminding the patient to take medications based on the patient medical data.
